(19) United States
(12) Patent Application Publication
(10) Pub. No.: US 2024/0285908 A1
(43) Pub. Date: Aug. 29, 2024

(54) ACCESSORY DEVICE FOR NAVIGATING CATHETERS

(71) Applicant: Maduro Discovery, LLC, Campbell, CA (US)

(72) Inventor: Brian B. MARTIN, Santa Cruz, CA (US)

(73) Assignee: Maduro Discovery, LLC, Campbell, CA (US)

(21) Appl. No.: 18/586,076

(22) Filed: Feb. 23, 2024

Related U.S. Application Data
(60) Provisional application No. 63/486,503, filed on Feb. 23, 2023.

(51) Int. Cl. A61M 25/01; A61M 25/09

(52) U.S. Cl. CPC A61M 25/0155; A61M 25/09; A61M 2210/12

(57) ABSTRACT

Catheter advancement assemblies that allow for a force applied at a distal portion of the catheter to drive the catheter from a location within the anatomy.

### CROSS-REFERENCE TO RELATED APPLICATIONS

This application is a non-provisional of U.S. Provisional Application No. 63/486,503 filed Feb. 23, 2023. The entirety of which is incorporated by reference.

### FIELD OF THE INVENTION

Catheter advancement assemblies that apply a force at a portion of the catheter to push or pull the catheter through anatomy by the application of the force in a geometrically advantageous or favorable position instead of, or in addition to, pushing the catheter from a proximal end.

### BACKGROUND OF THE INVENTION

Medical catheters allow physicians to apply a variety of different therapies within the body of a patient. Many catheters access remote regions of the human body for delivering diagnostic or therapeutic tools and/or agents to those sites. Alternatively, the catheter can comprise a shaft or support for a therapeutic working end (e.g., balloon, filter retriever, electrode, etc.). Some catheters, including but not limited to catheters for neurovascular use, are intended to be advanced from a main artery (e.g., a femoral or radial artery) through tortuous anatomy into a small cerebral vessel. As such, the catheter must be configured with varying structural traits due to the varying regions of the anatomy through which the catheter passes. Many times, the vascular pathways wind back upon themselves in a multi-looped path making it difficult for catheter design to meet the requirements demanded by the tortuous anatomy. For example, catheters must be fairly stiff at their proximal end so as to allow the pushing and manipulation of the catheter as it progresses through the body, and yet must be sufficiently flexible at the distal end to allow passage of the catheter tip through smaller blood vessels.

Additionally, in most cases, when a push force is applied at the proximal end of a catheter, that force is not efficiently translated to the distal end of the catheter. As the anatomy gets more and more tortuous, the efficiency of applied push force decreases because portions of the catheter between the proximal and distal ends encounter resistance due to the tortuous anatomy. Acute tortuous bends, such as bends and angulations that come from the major branching vessels off the aortic arch, and the often highly tortuous vessels in the carotid arteries reduce the push force that is translated to the distal end of the catheter. In these situations, the applied push force at the proximal end of a catheter can be significantly diminished, with the result being that very little of that push force applied to the proximal end of the catheter actually translates to the distal end of the catheter.

FIG. 1A illustrates an access catheter 10 being advanced over a guidewire 40 that is positioned through the vasculature. Typically, the proximal portion 52 and hub 54 of the catheter 50 enter the body through a remote region. For example, in order to advance a catheter into vessels located in the brain, a guide catheter is first introduced into an insertion site at a radial or femoral vessel and advanced through the aortic arch and into a right common carotid artery. Typically, a force 20 is applied to the proximal portion 52 or the hub 52 at the insertion site. This force, in turn, causes a driving force 22 in the catheter 50. As the catheter 50 navigates a turn or bend (e.g., as in the aortic arch 10), the resultant applied force 22 is distributed between components in the x-direction (Fx) and in the y-direction (Fy). Therefore, the greater the tortuosity or bend, the amount of component force Fx increases, which reduces component force Fy. As shown in FIG. 1B, as the catheter navigates further bends or tortuous paths (such as in branching vessel 12), the component Fx of driving force 22 again increases, thereby reducing the amount of component Fy, which moves the catheter 50 in the direction of desired travel. In some cases, if the component force (e.g., Fx) increases beyond a threshold, then the catheter 50 can no longer advance or simply folds/kinks due to the component force (e.g., Fx).

Conventional attempts to address this issue have focused on catheter design to allow force to be transmitted in the desired direction of travel. However, such configurations can increase the thickness of the catheter walls, which increases the outer diameter of the catheter or reduces the internal diameter of the catheter lumen. Moreover, such configurations prevent the catheter from accessing small, distally located vessels.

Therefore, there remains a need to improve the ability of a medical practitioner to advance a catheter through tortuous anatomy.

### SUMMARY OF THE INVENTION

The present disclosure includes advancement or navigation-assist assemblies for catheters and similar medical devices that allow the application of a navigation force at any region of the catheter located within the vessel, which allows application of the navigation force at a region of the catheter that is geometrically advantageous for navigation of the catheter. Therefore, a physician can choose to push or pull the catheter from the most strategically advantaged portion of the catheter given the position within the vessel. Such an advancement assembly can push and/or pull the catheter at any region of the catheter rather than only pushing the catheter from a proximal end. It is noted that although the present disclosure discusses use of the advancement assemblies to navigate a catheter to a desired location, the advancement navigation assemblies can also be used to reposition or withdraw a catheter.

In one variation, the present disclosure includes a method of navigating a catheter through a vascular region by inserting the catheter into a vessel; inserting a catheter advancement assembly into the catheter, the catheter advancement assembly comprising a shaft slidably located within a sheath, the shaft comprising at least one expandable member at a distal section; positioning the at least one expandable member in the interior of the catheter; inflating the at least one expandable member to expand against the interior of the catheter at a region of the catheter within the vessel; and moving a proximal section of the shaft relative to the sheath to cause movement of the distal section of the shaft, wherein a fit of the at least one expandable member against the interior the catheter applies a force at the region of the catheter within the vessel that causes movement of the catheter to further navigate the catheter within the vessel.

Another variation includes a method of navigating a catheter through a vascular region by inserting a catheter advancement assembly into the catheter, the catheter advancement assembly comprising a shaft slidably located within a sheath, the shaft comprising at least one expandable member at a distal section; positioning the at least one expandable member in an interior of the catheter; inflating the at least one expandable member to expand against the interior of the catheter at a region of the catheter located within the vessel; distally advancing a proximal section of the shaft relative to the sheath to cause distal advancement of the distal section of the shaft, wherein a fit of the at least one expandable member against the interior of the catheter also causes distal advancement of the catheter from the region of the catheter located within the vessel to further navigate the catheter within the vessel.

Another variation of a method can include advancing a catheter within a vascular region by inserting the catheter into a vessel; inserting a catheter distal advancement assembly into the catheter, the catheter distal advancement assembly comprising a shaft having at least one expandable member at a distal section; expanding the at least one expandable member against an interior of the catheter in a region of the catheter within the vessel; applying a first force at a proximal section of the shaft to cause the at least one expandable member to apply a second force at the region of the catheter within the vessel, where the second force causes movement the catheter to navigate the catheter within the vascular region.

The present disclosure also includes catheter, where the term catheter or catheters includes but is not limited to sheaths, introducers, medical tubing, and/or any device having a tubular portion that is used to deliver a working end, substances, or other devices to a site within the anatomy. Furthermore, the construction features of the improved connector are not limited to in-dwelling medical devices and can be used for any device requiring tubing.

For example, such a catheter can include a tubular structure comprising a lumen; a shaft slidably located within a sheath, the shaft comprising a first expandable member spaced from a second expandable member such that a portion of the shaft located therebetween can bend to allow the first expandable member to deflect independently of the second expandable member when expanded, the sheath configured for advancement within the catheter lumen, such that when inserted into a vessel the shaft assembly can be advanced distally from the sheath to expand the at least one expandable member within the catheter lumen wherein advancement of the shaft assembly relative to the sheath pulls a portion of the catheter within the vessel.

Variations of such catheters can comprise a thin wall catheter, a guide wire, or any other accessory or guiding device used with medical procedures.

The devices of the present disclosure allow for a considerable number of combinations and permutations of different variations of catheters as well as a combination of aspects of those structures as well. It is contemplated that any of the requirements and elements described herein can be combined with any independent claim where the requirements of the independent claims would not contradict the various elements.

### BRIEF DESCRIPTION OF THE FIGURES

FIGS. 1A and 1B illustrates a traditional catheter navigated within tortuous anatomy.

FIG. 1B illustrates the catheter of FIG. 1A encountering a vessel wall.

FIG. 2 illustrates an improved catheter advancement assembly located within a catheter where the advancement assembly.

FIGS. 3A and 3B illustrate variations of an improved catheter advancement assembly that allows for pushing or pulling of the catheter through a vascular region by applying a force on the catheter at a geometrically favorable region of the catheter.

FIG. 4A illustrates the shaft, balloon assemblies, and catheter moved distally within a branched vessel such that the assembly pushes the catheter at a distal region, which also pulls the remainder of the catheter from that distal region.

FIG. 4B illustrates movement of the catheter advancement assembly to a different region of the catheter.

FIGS. 5A to 5E show another aspect of a catheter advancement assembly used to sequentially advance a catheter.

FIG. 6 illustrates another variation of a catheter advancement assembly with a distal expandable member that can fully or partially extend from a tip of the catheter to assist in navigation of the catheter.

FIG. 7 shows the catheter positioned in the anatomy with the catheter advancement assembly removed from the catheter and body.

FIGS. 8A to 8C show additional variations of multiple layer balloons for use with any variation of the devices described herein.

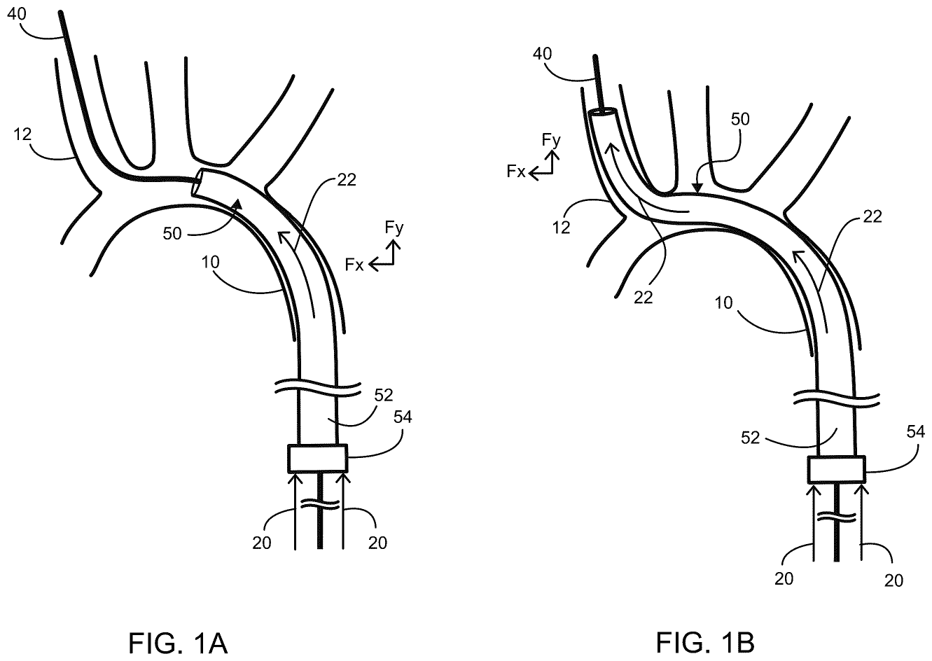

### DETAILED DESCRIPTION

The catheter configuration discussed herein can be used in a variety of devices that need to be advanced through tortuous anatomy. The configurations described herein can be incorporated into various tubular medical devices similar to catheter shafts. Furthermore, in some variations, the construction features of the present disclosure are not limited to in-dwelling medical devices and can be used for any device requiring improved navigation.

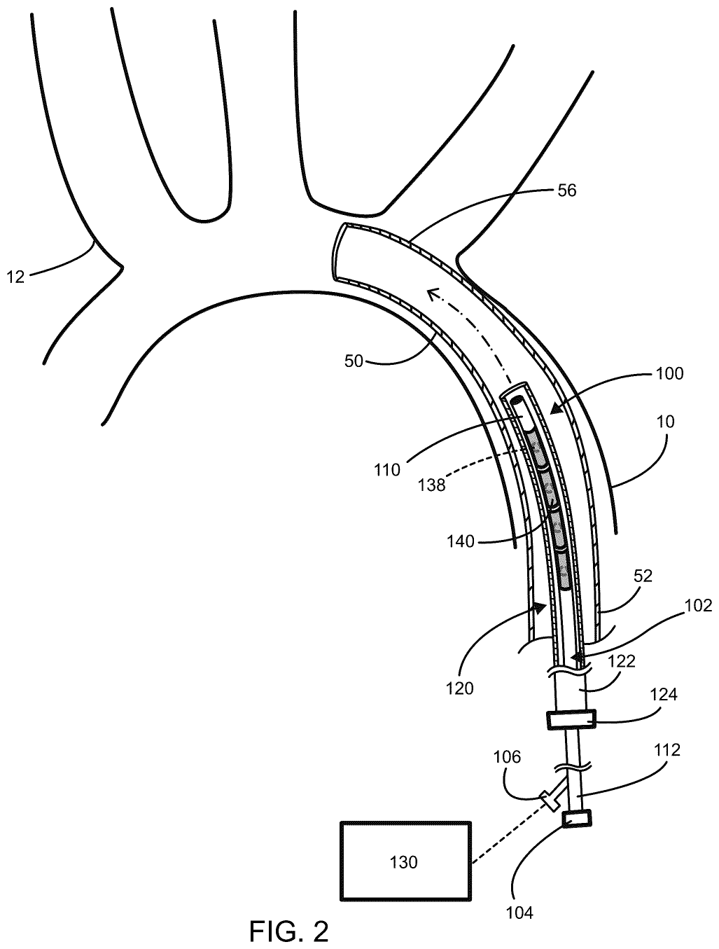

FIG. 2 illustrates an improved catheter advancement assembly 100 located within a catheter 50, such as an access catheter, where the advancement assembly 100 includes a shaft 102 slidably located within a sheath device 120. Although not illustrated, the advancement assembly 100 can include a lumen to allow advancement of the assembly 100 over a guide-catheter or guidewire. Moreover, the catheter advancement assembly 100 can be used after the access catheter 50 is already positioned within a vessel 10. Alternatively, the catheter advancement assembly 100 can be positioned within the catheter 50, and then the catheter 50 containing the assembly 100 can be inserted into the vasculature such that the proximal end 52 of the access catheter 50 extends externally from a body of a patient (e.g., through a radial or femoral artery). Regardless, an expandable structure assembly 40 of the catheter advancement assembly 100 is ultimately advanced towards a distal region 56 of the catheter 50, as discussed in further detail below.

The expandable structures described herein can include compliant or non-compliant inflatable balloon materials. In one embodiment, one or more balloons are comprised of two or more layers, where the inner layer is a non-compliant material (PET, nylon) for maximizing inflation force and applying high force to the inside of the catheter body, covered by a compliant material (i.e., urethane, silicone, etc.) for maximizing the frictional forces. Additionally, the device can comprise the expandable mechanical structure(s), such as braid, stent, nitinol scaffold, etc., which can expand and contract similar to the inflatable balloon. In yet additional variations, the expandable structure can include a combination of expandable structures such as balloons, braid, stent, nitinol scaffold, etc.

As shown in FIG. 2, the advancement assembly 100 includes one or more expandable structures 140. Variations of the advancement assembly 100 that include balloons can comprise segmented balloons (either a single balloon segmented into sections or multiple balloons). A segmented balloon 40 configuration allows the shaft 102 and balloon(s) 140 to flex during navigation when advanced through the anatomy, as discussed below. The balloon(s) 140 include a port 138 that provides a fluid path to one or more ports 106. The port or ports 106 allows the balloon assembly 140 to be fluidly coupled to a fluid source 130. Once located within the access catheter 50, the shaft 102 of the catheter advancement assembly 100 advances toward the distal region of the access catheter 50. As shown, a proximal section 112 of the shaft 102 can include a handle or hub 104 that allows for passage of a guidewire. The proximal section 112 and/or hub 104 can also be moved by securing a proximal portion 122 of the sheath 120 and/or a sheath hub 124 and advancing the proximal section 112 of the shaft to position a distal section 110 of the shaft 102 adjacent to the distal region 56 of the catheter 50. Although not shown, additional variations of the catheter advancement assembly 100 can omit the sheath 120, where the shaft 102 is advanced directly within the access catheter 50 with or without a guidewire. The shaft 102 of the assembly 100 can comprise any reinforcement structure to increase column strength to allow for advancing of the catheter.

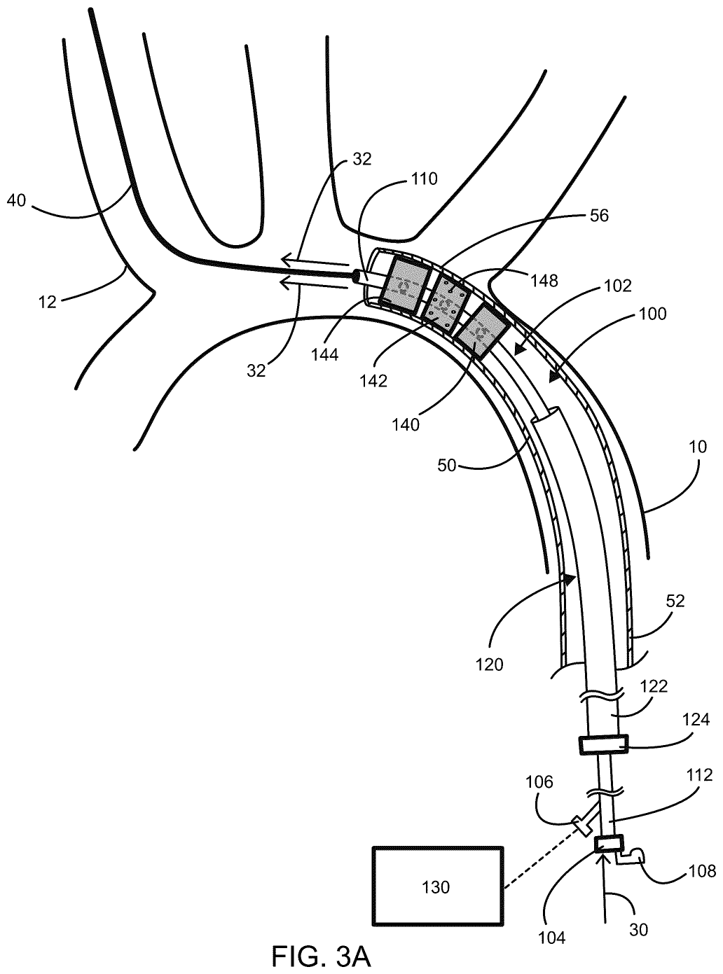

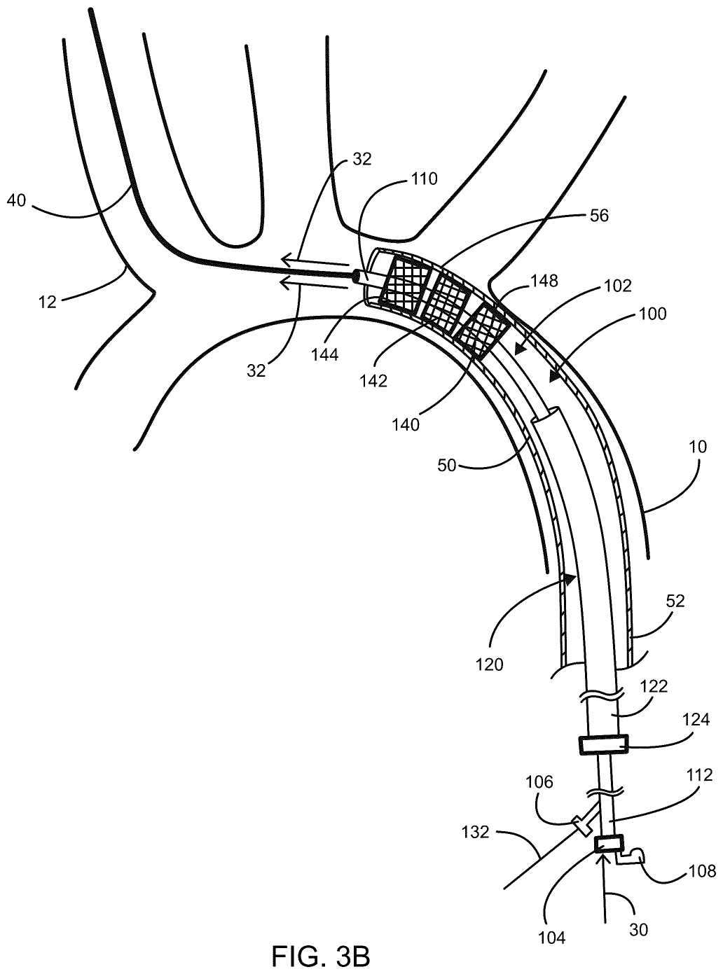

FIG. 3A illustrates another variation of an improved catheter advancement assembly 100 that, when deployed in the access catheter 50, allows for pulling of the catheter 50 (shown in a cross-sectional view) through a vascular region by the application of a force at a location that is proximal to the catheter 50 (e.g., a radial or femoral artery as noted above). In the variation shown in FIG. 3A, the catheter advancement assembly 100 includes a shaft that is configured to slide relative to a sheath 120. The variation shows the assembly 100 advanced over a guidewire 40 that passes to an intended location within a branched vessel 12. However, the use of a guidewire is optional. Some variations of the assembly 100 include steering mechanisms and steering controls 108 that allow navigation of the shaft 102 or deflection of the distal section 110 of the shaft with or without the use of a guidewire.

FIG. 3A shows a balloon assembly comprising one or more expanded balloon members 140, 142, 144 located at a distal section 110 of the shaft 102. Again, additional variations of the shaft 102 can include a single balloon that is segmented to form multiple sections (e.g., 140, 142, 144). Alternatively, the shaft 102 can include multiple balloons. The use of multiple or segmented balloons allows for the balloons and shaft 102 to conform or flex when advanced through tortuous anatomy. The balloons 140, 142, 144 can be coupled to one or more fluid ports 106 that can be further coupled to a fluid source 130 (e.g., a syringe of liquid, an air/gas source, etc.). In the case of separate balloons, each balloon, or groups of balloons, can be independently expandable from the remaining balloons. Moreover, while the illustration shows three balloons, any number of balloons is within the scope of the disclosure. It is understood that the assembly 100 can include a single balloon towards a distal portion, or multiple balloons as discussed above. Moreover, one or more balloons can be positioned on a proximal portion of the shaft 102.

As shown in FIG. 3A, the surface of any of the balloons disclosed herein can be optionally treated 148 to increase a grip or friction between the interior of the access catheter 50 and the balloon surface. Alternatively, or in addition, the surface of the balloon can include mechanical structures that interact with mechanical features on the interior of the access catheter.

FIG. 3A also illustrates assembly as a two-piece system with distal balloons 140, 142, 144 and a sheath 120 proximal to the balloons. In the variation shown, the shaft 102 is advanced independently of the sheath 120. Therefore, when the sheath 120 is positioned within the catheter 50, the shaft 102 can be advanced out of the sheath 120 to position the distal section 110 of the shaft 102 adjacent to a distal region 56 of the access catheter 50 for inflation of the balloons 140, 142, 144, which releasably locks the distal section 110 of the shaft 102 to the distal region 56 of the catheter 50. Once locked together, the caregiver can hold the proximal portion 122 of the sheath 102 (or the sheath hub 124) while applying a distally directed force 30 on the proximal section 112 of the shaft 102. The distally directed force 30 causes distal movement of the distal section 110 of the shaft 102, and because the shaft 102 is locked to the distal region 56 of the access catheter 50, movement of shaft 102 and balloons 140, 142, 144 pushes the distal region 56 of the access catheter 50 to advance it through the vessel. This also pulls the remainder or proximal end 52 of the access catheter 50 further into the vessel 10 or 12. This action creates a distal driving-effect for navigation and advancement of the catheter 50 through the vasculature, where force is applied at the distal region 56 and a force can optionally be applied to the proximal portion 52 of the catheter to navigate the catheter 50 through the anatomy.

It is noted that variations of the device 100 include a shaft 102 without any outer sheath 120. In such variations, the shaft 102 advances by itself or via navigation over a guidewire.

FIG. 3B illustrates a similar device 100 where the expandable structures comprise one or more mechanical structures 140, 142, 144, including but not limited to a braid, stent, nitinol scaffold, etc. Expansion and contraction of one or more of the mechanical structures 140, 142, 144 can occur via one or more wires/lines 132 and/or through a push/pull actuator or other means generally known in such associated technologies.

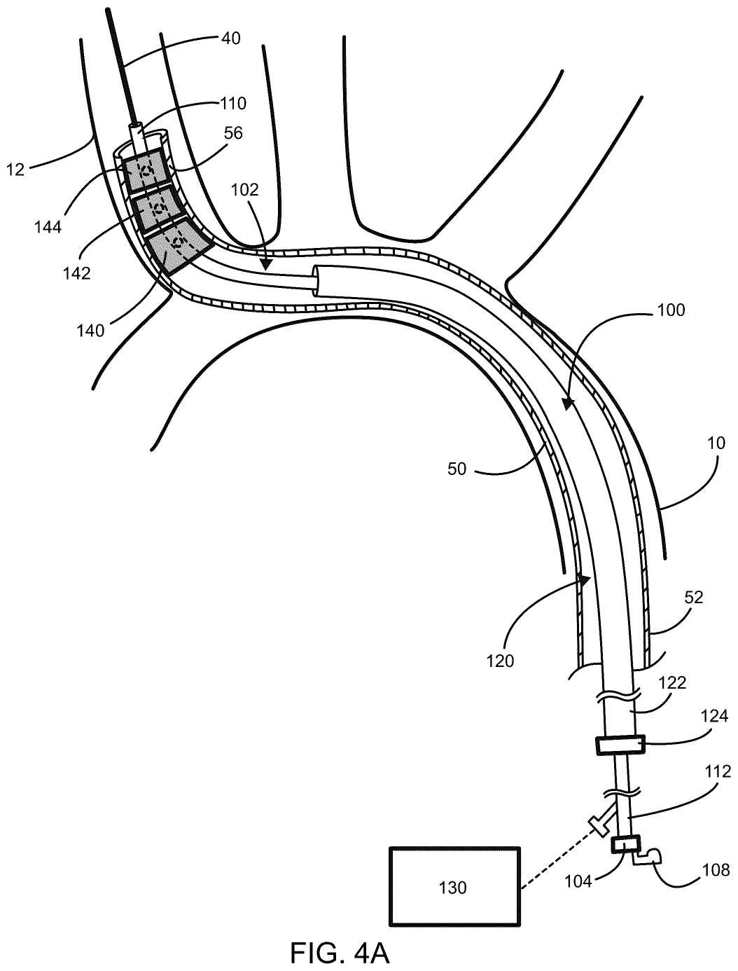

FIG. 4A illustrates a state where the shaft 102 and balloon assemblies 140, 142, 144 are expanded against the interior of the distal region 56 of the catheter 50 and moved distally within the branched vessel 12 such the shaft 102 and balloon assemblies 140, 142, 144 push the catheter 50 at the distal region 56 and pull the remainder of the catheter 50. As discussed above, advancement of the shaft 102 can occur over a guidewire/guide catheter 40 or via steering of the tip through controls 108 at the proximal portion 112 or hub 104 of the shaft 102. This driving of the catheter 50 at the distal portion is far more advantageous and more efficient than the traditional approach of pushing the catheter at the extreme proximal end.

In the illustration of FIG. 4A, the sheath 120 advances from the position shown in FIG. 3A. Accordingly, the catheter advancement assembly 100 can drive an access catheter 50 by advancement of the shaft 102 without advancement of the sheath 120 or by repeated incremental advancement of the shaft 102 and then the sheath 120. In an additional variation, the sheath 120 can remain in place, while the balloons and shaft 102 assembly are cycled multiple times proximally and distally to advance the catheter 50. However, any number of combinations are within the scope of this disclosure that allows for navigational advancement of an access catheter 50 by applying a force at a distal region 56 to push the distal region 56 and pull the remainder of the catheter 50 through the anatomy.

As noted above, the use of the assembly to drive the catheter through the vasculature results in a more efficient transfer of force to advance the catheter. Variations of the methods and systems described herein can also include one or more expandable structures 140, 142, 144 deployed at any portion of the catheter 50. As shown in FIG. 4A, one or more expandable structures 140, 142, 144 are deployed at a distal region 56 of the catheter 50. However, as shown in FIG. 4B, one or more expandable structures 140, 142, 144 can deployed at any other region of the catheter 50 between a distal region 56 and a proximal region 52 to apply a driving force at that region to assist in advancing or withdrawing of the catheter 50. As shown, the expandable structures are positioned in an intermediate region 58. Moreover, the expandable structures can be repositioned within the catheter 50 as needed for proper positioning of the catheter within the vessel 10 or branching vessel 12.

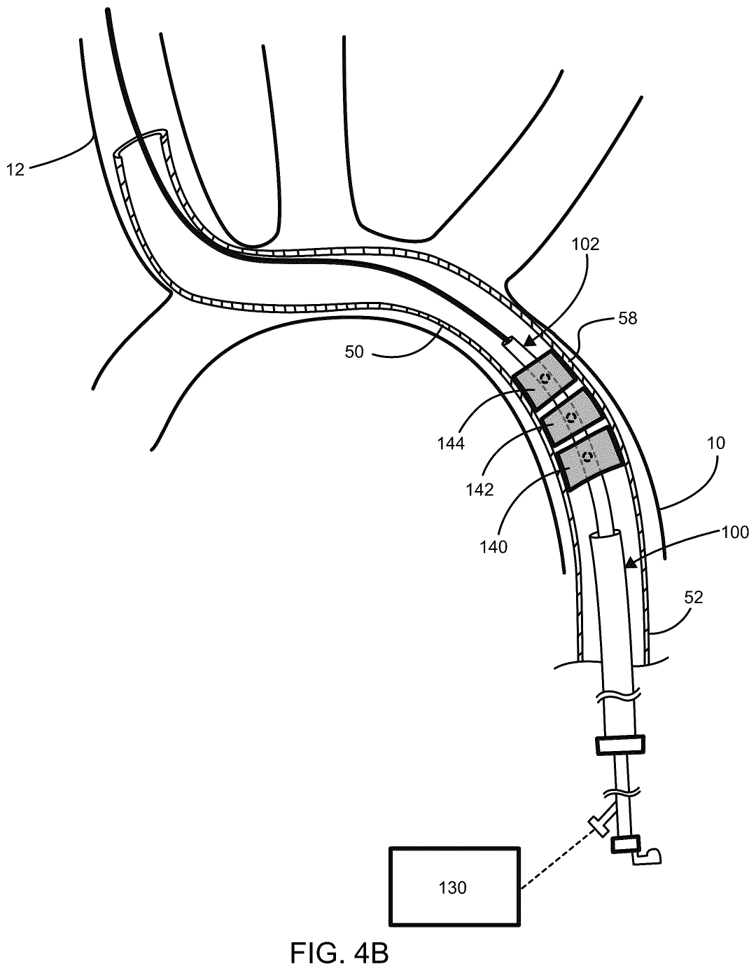

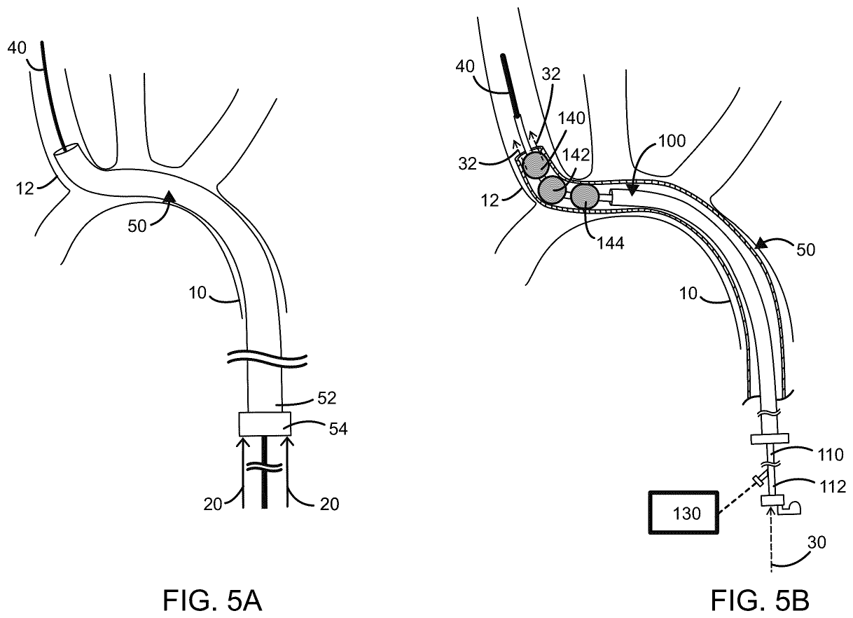

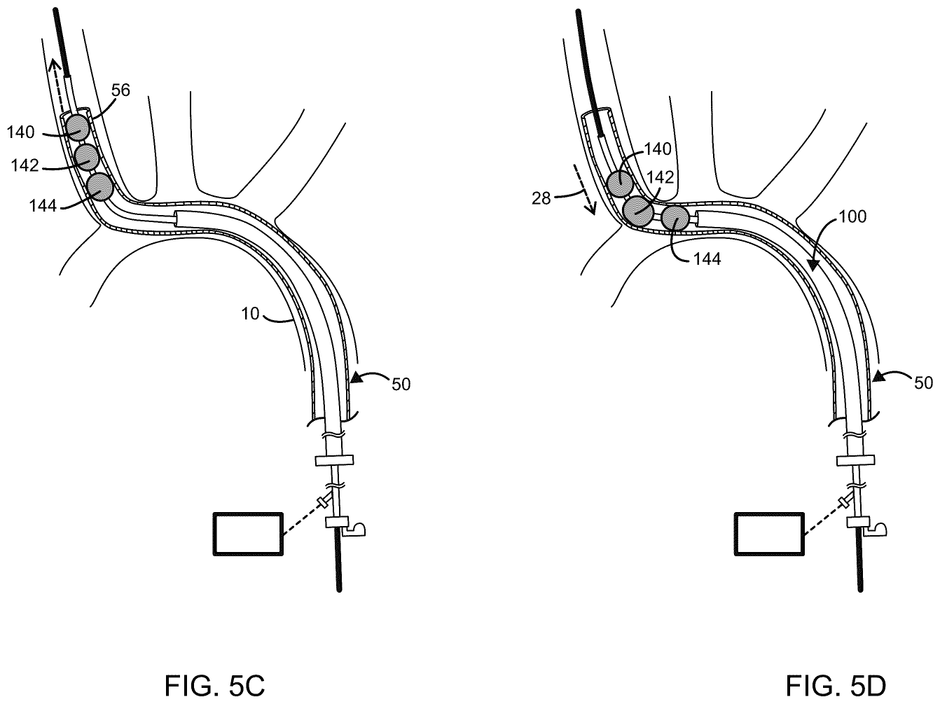

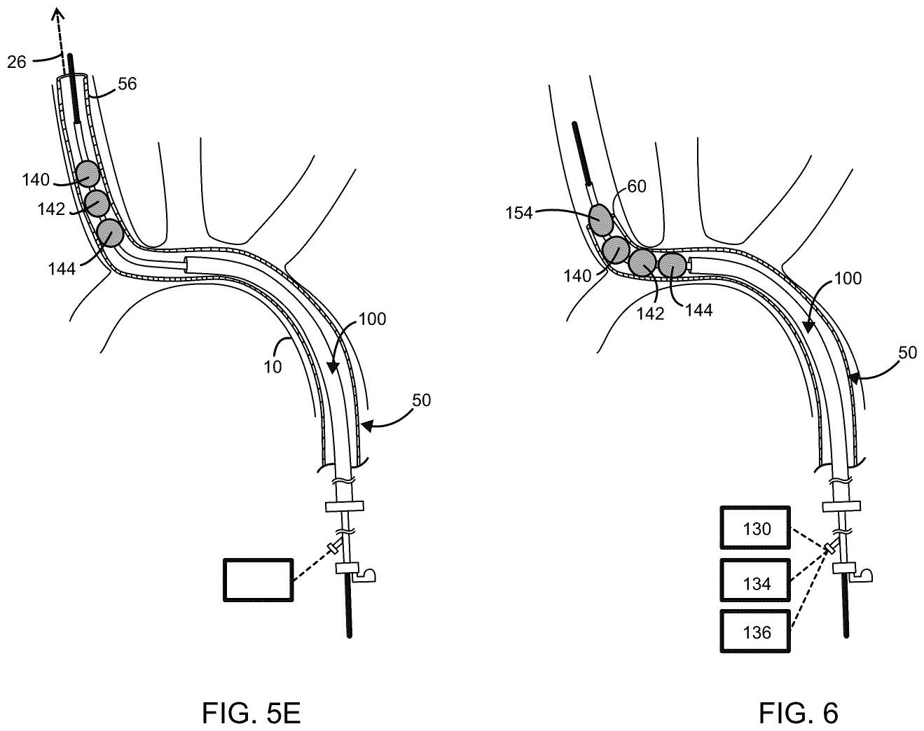

FIGS. 5A to 5E show another aspect of a catheter advancement assembly as disclosed herein. FIG. 5A represents a catheter 50 that is navigated through a vessel 10 to a bends or tortuous paths (such as in branching vessel 12) during a conventional procedure where a pushing force 20 is applied to a proximal end 52 or hub 54 of the catheter 50. In many cases, further navigation of the catheter 50 in the branching vessel 12 is difficult due to the tortuosity of the anatomy.

FIG. 5B illustrates a partial cross-sectional view of the catheter 50 of FIG. 5A with a catheter advancement assembly 100 positioned within the catheter 50. As shown, the expandable members 140, 142, 144 can be expanded as discussed herein against a portion of the catheter 50 that is located within the vessel 10. The illustration shows the expandable members 140, 142, 144 positioned in a region of the anatomy that gives a geometric advantage in applying a force to the catheter 50 for navigation. In this example, a force pushing force 30 at the proximal end of the shaft 110 results in the expandable members 140, 142, 144 applying that force 32 at the region of the catheter 50 in contact with the expandable members 140, 142, 144, which is within the vessel 10 and at a location that provides a geometric benefit (e.g., the bifurcation or tortuosity). As discussed herein, this causes the catheter 50 to be pushed at the distal region, which simultaneously pulls the proximal region.

FIG. 5C illustrates the expandable members 140, 142, 144 moving the catheter 50 through the vessel 10 to advance the distal region 56 to a desired target site. Once advanced, as shown in FIG. 5D, the expandable members 140, 142, 144 can be withdrawn in direction 28 within the catheter 50 while the catheter 50 remains stationary. FIG. 5E shows the repositioned expandable members 140, 142, 144 of FIG. 5D further advancing within the vessel 10 to further advance the distal region 56 in direction 26. This sequential movement shown in FIGS. 5B to 5E can be repeated as needed. In addition, this sequential advancement can be performed in any portion of the catheter 50 with or without applying a pushing force at the proximal end of the catheter 50 as shown in FIG. 5A.

FIG. 6 illustrates an additional variation of a catheter advancement assembly 100 for navigation of a catheter 50 where the assembly 100 includes one or more expandable members 140, 142, 144 along with a distal expandable member 154 that can extend totally or partially from a distal tip 60 of the catheter 50. This distal expandable member 154 can comprise a tapered or atraumatic shape or any rounded shape that prevents the tip 60 of the catheter from getting caught on surfaces or bifurcations as the catheter 50 advances through the anatomy. As discussed herein, the assemblies 100 of the present disclosure can include any number of expandable members or can include a single expandable member that is segmented to allow flexibility to assist in navigating the catheter 50 within the anatomy. Moreover, the expandable members can be selectively/individually expanded via one or more sources 130, 134, 136.

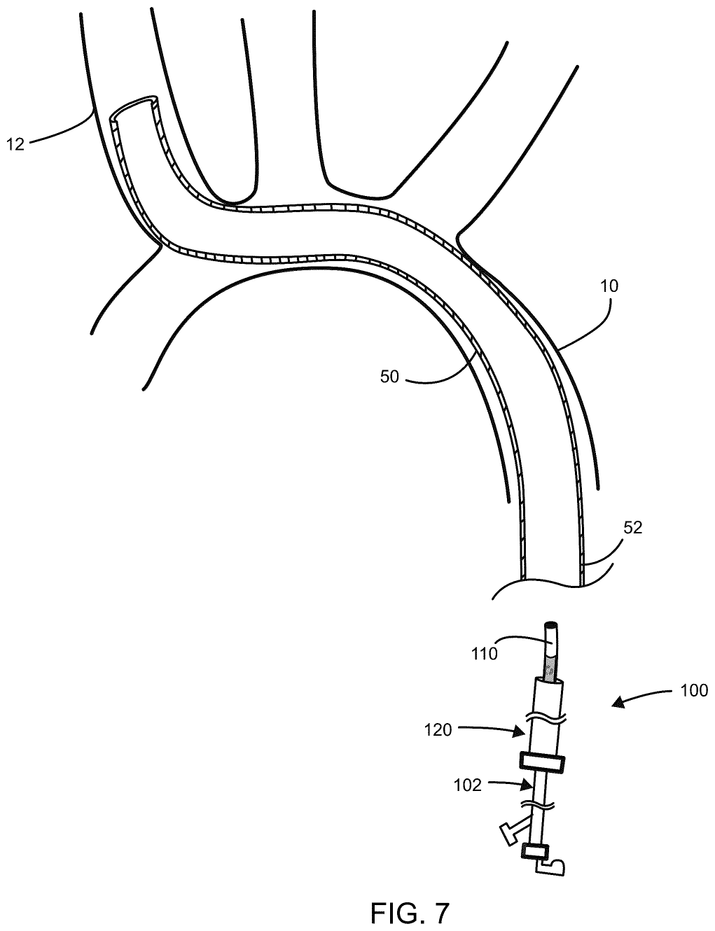

As shown in FIG. 7, once the catheter 50 reaches an intended target site, the balloon assembly 140, 142, 144 can be deflated and the catheter advancement assembly 100 is withdrawn from the proximal end 52 of the catheter 50 as well as removed from the vessel/anatomy. The catheter 50 can remain deployed within the vessel 10 and/or branching vessel 12.

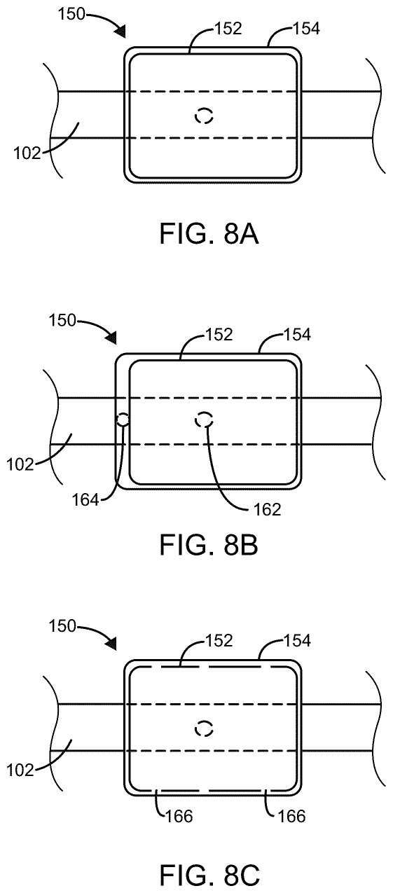

FIGS. 8A to 8C illustrate additional variations of balloon or expandable members 150 that can be used in any of the variations discussed herein (e.g., expandable members 140, 142, 144, 154). In this variation, as shown in FIG. 8A, the balloon member 150 can comprise a composite construction with an interior balloon 152 that comprises a non-distensible/non-compliant material. Such materials can comprise a high durometer material, PET, Nylon, etc.). Therefore, during expansion of the inner balloon 152, the balloon 152 expands to a high pressure inflation condition with a fixed shape. Next, the exterior balloon member 154, which comprises a low durometer, material, e.g., urethane, silicone, etc. provides a tacky or high friction surface for maximum grip against the interior of the catheter. FIG. 8B illustrates a condition where an inflation lumen 162 for the interior balloon member 152 is separate from the exterior balloon 154, which has a fluidly isolated inflation lumen 164 from the inner balloon member 152. FIG. 8C shows a variation where the interior balloon member 152 includes openings or other ports 166 that allow for inflation of the exterior balloon member 154.

As for other details of the present invention, materials and manufacturing techniques may be employed within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

1. A catheter driving assembly for use with a catheter, the catheter driving assembly comprising:
a sheath comprising a lumen;
a shaft slidably located within the sheath, the shaft comprising a first expandable member spaced from a second expandable member such that a portion of the shaft located therebetween can bend to allow the first expandable member to deflect independently of the second expandable member when expanded, the sheath configured for advancement within the lumen, such that when inserted into a vessel the shaft can be advanced distally from the sheath allowing expansion of the first expandable member and/or the second expandable member within an interior of a catheter located in the vessel and wherein advancement of the shaft relative to the sheath applies a driving force at a region of the catheter within the vessel to advance the catheter within the vessel.

2. The catheter driving assembly of claim 1, wherein the catheter comprises a thin wall catheter.

3. The catheter driving assembly of claim 1, further comprising a guide wire.

4. The catheter driving assembly of claim 1, further comprising at least a third expandable member spaced from the second expandable member.

5. The catheter driving assembly of claim 1, further comprising a distal expandable member that is configured to extend from a distal tip of the catheter, where the distal expandable member comprises a curved distal surface.

6. The catheter driving assembly of claim 1, further comprising a fluid source coupled to the first expandable member.

7. The catheter driving assembly of claim 6, wherein the second expandable member is coupled to a second fluid source.

8. The catheter driving assembly of claim 7, further comprising a steering control configured to deflect or steer a distal end of the shaft.

9. The catheter driving assembly of claim 1, wherein one or more of the expandable members is expandable and contractable using an actuator.

10. The catheter driving assembly of claim 1, wherein at least one expandable member comprises a dual layer balloon having an inner inflation member and an outer inflation member exterior to the inner inflation member, where a durometer of the outer inflation member is less than a durometer of the outer inflation member such that the expandable member increases friction against the catheter when expanded.

11. A method of navigating a catheter through a vascular region, the method comprising:
inserting the catheter into a vessel;
inserting a catheter advancement assembly into the catheter, the catheter advancement assembly comprising a shaft slidably located within a sheath, the shaft comprising at least one expandable member at a distal section;
positioning the at least one expandable member in an interior of the catheter;
inflating the at least one expandable member to expand against the interior of the catheter at a region of the catheter within the vessel; and
moving a proximal section of the shaft relative to the sheath to cause movement of the distal section of the shaft, wherein a fit of the at least one expandable member against the interior the catheter applies a force at the region of the catheter within the vessel that causes movement of the catheter to further navigate the catheter within the vessel.

12. The method of claim 11, wherein the region of the catheter within the vessel comprises a distal region of the catheter.

13. The method of claim 11, wherein the region of the catheter within the vessel comprises a mid region of the catheter located between a distal region and a proximal region of the catheter.

14. The method of claim 11, wherein positioning the at least one expandable member comprises positioning the at least one expandable member in a proximal region of the catheter.

15. The method of claim 11, further comprising repositioning the at least one expandable member in a second location in the catheter and moving the proximal section of the shaft relative to the sheath to cause movement of the distal section of the shaft, wherein the fit of the at least one expandable member against the interior the catheter at the second location applies the force at the second location to cause further movement of the catheter to navigate the catheter within the vessel.

16. A method of navigating a catheter that is positioned in a vascular region of a vessel, the method comprising:
inserting a catheter advancement assembly into the catheter, the catheter advancement assembly comprising a shaft slidably located within a sheath, the shaft comprising at least one expandable member at a distal section;
positioning the at least one expandable member in an interior of the catheter;
inflating the at least one expandable member to expand against the interior of the catheter at a region of the catheter located within the vessel; and
distally advancing a proximal section of the shaft relative to the sheath to cause distal advancement of the distal section of the shaft, wherein a fit of the at least one expandable member against the interior of the catheter also causes distal advancement of the catheter from the region of the catheter located within the vessel to further navigate the catheter within the vessel.

17. The method of claim 16, wherein positioning the at least one expandable member in an interior of the catheter occurs prior to insertion of the catheter within the vascular region.

18. The method of claim 16, wherein positioning the at least one expandable member in an interior of the catheter occurs after insertion of the catheter within the vascular region.

19.-24. (canceled)
